(19) United States
(12) Patent Application Publication
(10) Pub. No.: US 2019/0021669 A1
(43) Pub. Date: Jan. 24, 2019

(54) Methods, Apparatuses, and Systems for Monitoring Biomechanical Behaviors

(71) Applicants: FNU Apoorva, Ithaca, NY (US); Jason Guss, Ithaca, NY (US); Pankaj Singh, Ithaca, NY (US)

(72) Inventors: FNU Apoorva, Ithaca, NY (US); Jason Guss, Ithaca, NY (US); Pankaj Singh, Ithaca, NY (US)

(21) Appl. No.: 16/041,172

(22) Filed: Jul. 20, 2018

Related U.S. Application Data
(60) Provisional application No. 62/534,784, filed on Jul. 20, 2017.
(60) Provisional application No. 62/673,664, filed on May 18, 2018.

(51) Int. Cl. A61B 5/00; G06Q 10/06; G01L 5/00; G01P 13/00; G01P 15/08; G01K 7/22; A61B 5/0492

(52) U.S. Cl. CPC A61B 5/6806; A61B 2090/064; G01L 5/00; G01P 13/00; G01P 15/0802; G01K 7/22; A61B 5/0492; A61B 2503/20; A61B 2562/0209; A61B 2562/0219; A61B 2562/0276; A61B 2562/046; A61B 2562/066; A61B 2562/164; A61B 2562/166; G06Q 10/0635

(57) ABSTRACT

A device that may be used to monitor a user's biomechanical behavior to investigate the synergy of risk factors in the workplace. The device may assesses factors such as motion, force, vibration, and/or temperature experienced by a user for a period of time. Data may be uploaded from the device to software configured to assess safety and risk associated with the user's behavior. Modifications to the user's biomechanical behavior may then be modified to mitigate safety risks.

### CROSS REFERENCE TO RELATED APPLICATIONS

This application claims the benefit of U.S. Provisional Patent Application No. 62/534,784, filed Jul. 20, 2017; and U.S. Provisional Patent Application No. 62/673,664, filed May 18, 2018. The entire contents of these applications are incorporated herein by reference.

### FIELD OF THE INVENTION

This invention relates to methods, apparatuses, and systems for monitoring biomechanical behaviors and exposure of workers to stress-related injuries, and identifying the synergies of work related risk factors.

### BACKGROUND OF THE INVENTION

Workplace injuries have become a larger focus in recent years as worker compensation costs have grown. In 2015, Repetitive Motion Injuries accounted for two-thirds of all work related injuries and cost corporations approximately $54 billion. The synergistic effect of risk factors that may lead to a Repetitive Motion Injury (RMI) has been studied, but no tools enabling the investigation of their combined effect have reached the market. Performing a risk assessment in the workplace is a time consuming and qualitative task with large implications, but the tools used for risk assessment have not developed over the past twenty years and the number of professionals trained to perform risk assessments have declined. There is therefore a need for improved methods of performing risk assessments that overcome some or all of the previously described drawbacks.

### SUMMARY OF THE INVENTION

The present invention is directed to a wearable device that may be used to perform risk assessments for worker behavior to identify and help address causes of RMIs. For example, a user interested in performing risk assessment on workers can use the device to collect data on the workers' biomechanical motions evaluate the synergy of risk factors such as repetitive motion, strain, and temperature. Multiple sensors may be incorporated into each processing unit on the hardware module of the smart wearable biomechanical behavior and exposure monitoring device to simultaneously measure mechanical forces, vibration, motion, temperature, muscle activity and other factors.

Numerous variations may be practiced in the preferred embodiment.

### BRIEF DESCRIPTION OF THE DRAWINGS

A further understanding of the invention can be obtained by reference to embodiments set forth in the illustrations of the accompanying drawings. Although the illustrated embodiments are merely exemplary of systems, methods, and apparatuses for carrying out the invention, both the organization and method of operation of the invention, in general, together with further objectives and advantages thereof, may be more easily understood by reference to the drawings and the following description. Like reference numbers generally refer to like features (e.g., functionally similar and/or structurally similar elements).

The drawings are not necessarily depicted to scale; in some instances, various aspects of the subject matter disclosed herein may be shown exaggerated or enlarged in the drawings to facilitate an understanding of different features. Also, the drawings are not intended to limit the scope of this invention, which is set forth with particularity in the claims as appended hereto or as subsequently amended, but merely to clarify and exemplify the invention.

FIG. 1 is a schematic diagram of a device in accordance with the present invention;

FIG. 2 is a schematic diagram of a software module in accordance with the present invention;

FIGS. 3A-3C depict possible motions that may be assessed by a device in accordance with the present invention;

FIGS. 4A and 4B depict top and bottom views of a device in accordance with the present invention;

FIG. 5 is a flowchart depicting steps in an illustrative embodiment of a method in accordance with the present invention;

FIG. 6 is a flowchart depicting steps in an illustrative embodiment of a method in accordance with the present invention;

FIGS. 7A-7D depict alternative configurations of devices in accordance with the present invention;

FIG. 8 is a flowchart depicting steps in an illustrative embodiment of a method in accordance with the present invention;

FIG. 9 is a table of exemplary risk scores;

FIG. 10 is a chart of risk factors that can lead to repetitive motion injuries;

FIG. 11 is a flowchart depicting transfer and processing of data an illustrative embodiment of a method in accordance with the present invention;

FIG. 12 is a flowchart depicting transfer and processing of data an illustrative embodiment of a method in accordance with the present invention;

FIG. 13 is a flowchart depicting transfer and processing of data an illustrative embodiment of a method in accordance with the present invention.

FIG. 14 depicts an exemplary location of an accelerometer in accordance with the present invention.

FIG. 15 depicts exemplary location of pressure sensors in accordance with the present invention.

FIG. 16 an exemplary layout of printed circuit board in accordance with the present invention.

### DETAILED DESCRIPTION OF THE INVENTION

The invention may be understood more readily by reference to the following detailed descriptions of embodiments of the invention. However, techniques, systems, and operating structures in accordance with the invention may be embodied in a wide variety of forms and modes, some of which may be quite different from those in the disclosed embodiments. Also, the features and elements disclosed herein may be combined to form various combinations without exclusivity, unless expressly stated otherwise. Consequently, the specific structural and functional details disclosed herein are merely representative. Yet, in that regard, they are deemed to afford the best embodiment for purposes of disclosure and to provide a basis for the claims herein, which define the scope of the invention. It must be noted that, as used in the specification and the appended claims, the singular forms “a”, “an”, and “the” include plural referents unless the context clearly indicates otherwise.

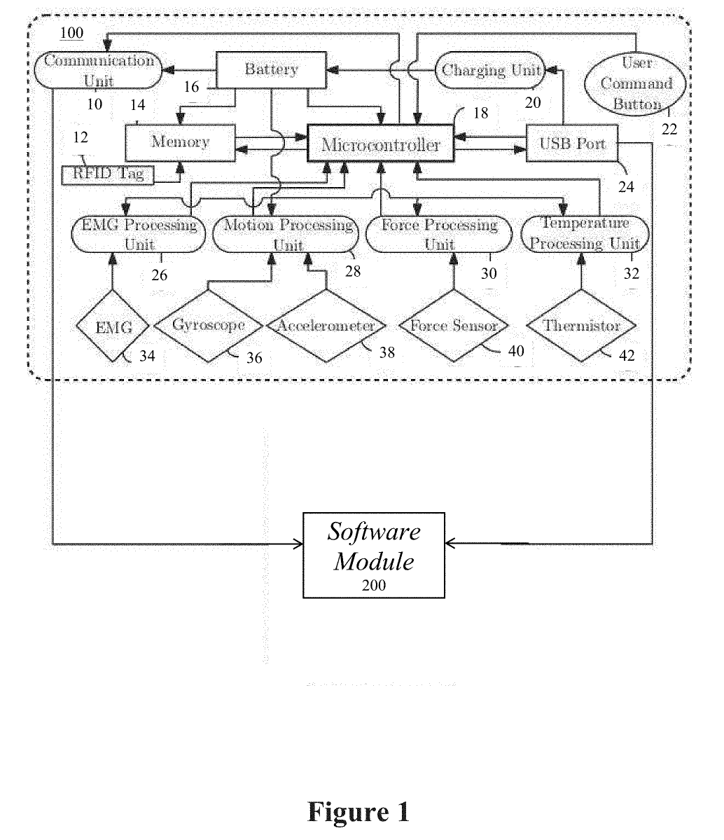

FIG. 1 depicts an exemplary schematic view of hardware module (100) of a device in accordance with the present invention. The device may be a smart, wearable, biomechanical behavior and exposure monitoring device. The device may be worn by a user, and may be used to measure the user's biomechanical behavior. For example, the device may be a glove or an armband. If the device is a glove, it may have a plurality of fingers, a thumb, and a palm. The finger of the glove furthest from the thumb of the glove is referred to herein as the pinky finger.

The device may be attached to or worn on a particular part of a user's body (e.g. a hand). One or more sensors of the device may be used to detect biomechanical behavior and external exposures to the user. The sensors may include one or more of: an electromyogram (EMG) sensor (34), a gyroscope (36), an accelerometer (38), a force sensor (40), and/or a thermistor (42). Although each of those types of sensors is shown in the device depicted in FIG. 1, a device in accordance with the present invention may have only one type of sensor, or a subset of those types of sensors. In addition, although hardware module (100) depicted in FIG. 1 has only one of each type of sensor, a device in accordance with the present invention may have a plurality of one or more of each type of sensor.

A device in accordance with the present invention may also have one or more other types of sensors. For example, the device may include one or more sensors such as blood pressure sensors, pulse oximeters, galvanic skin, strain gauge respiration sensors, and/or moisture sensors. Measurement data generated by any or all of the sensors may be recorded and stored in non-transitory memory (14).

Hardware module (100) may further consist of one or more processing units to record and/or process data. The processing units may include: a motion processing unit (28), a force processing unit (30), a temperature processing unit (32), and a muscular activity processing unit (26). The motion processing unit (28) may comprise a gyroscope (36) and/or an accelerometer (38). Gyroscope (36) and/or an accelerometer (38) each may be configured to monitor and record data on the motions of the user. Gyroscope (36) and accelerometer (38) may be used together to measure, for example, the orientation of the user's palm with respect to the wrist, and vibrations.

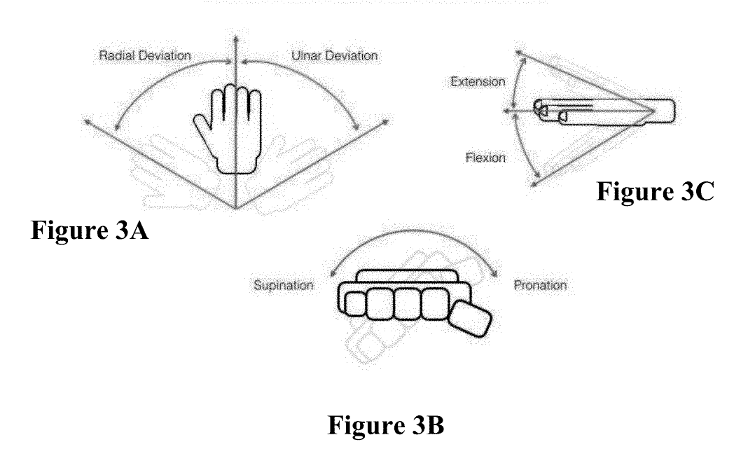

Force processing unit (30) may comprise one or more force sensors (40) that are able to monitor and record data on force applied by the user and force applied to the user. For example, to investigate worker risk of developing job-related injuries, motion data may be collected and analyzed to identify problematic movements. FIGS. 3A-3C depict examples of hand and wrist movements that can be identified, such as extension/flexion, radial deviation/ulnar deviation, and pronation/supination.

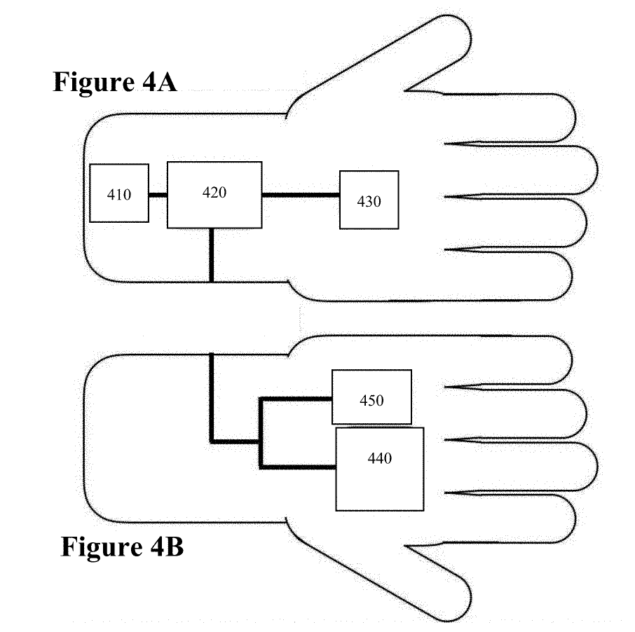

FIG. 4A depicts example placements of accelerometers (410, 430) that may be used to measure hand and wrist movements. FIG. 4A depicts placement of the accelerometers relative to the back of a user's hand. Circuit board (420) may send control signals to each accelerometer (410, 430) and receive data from each accelerometer (410, 430).

FIG. 4B depicts placement of force sensor (440) and temperature sensor (450) relative to the palm of a user's hand. Force sensor (440) may measure force transmitted through a user's palm. For example, to investigate hand and wrist biomechanical behavior, one or more force sensors (e.g., pressure sensors) may measure grip force applied by the hand and applied to the hand. FIG. 4B depicts an example location of force sensor (440) that me be used to measure grip force of the hand.

Extreme temperatures may also be a risk factor that, when experienced with poor biomechanical behavior or excessive force, may cause or contribute to an injury. A device according the present invention may include one or more temperature sensors (e.g., thermistors) (42, 450) configured to measure the temperature of a region of the body where the device is worn and/or the temperature of the environment. The temperature sensors (42, 450) may be part of a temperature processing unit (32).

Excessive or highly repetitive muscle activity may be another risk factor that may cause or contribute to an injury. The muscular activity processing unit (26) may include one or more electromyogram sensor(s) (34) that may be configured to monitor and record data on the muscle activity of the user of the device. Each electromyogram sensor (34) may measure the electric impulses of musculature and nerves. For example, to investigate worker risk of developing job-related injuries, muscle activity may be measured to determine whether it increases the development, progression, or severity of an injury.

A device in accordance with the present invention may have a microcontroller (18). Microcontroller (18) may be prompted by a user to initialize a motion processing unit (28), a force processing unit (30), a temperature processing unit (32), and/or a muscle activity processing unit (26). Microcontroller (18) may further record various biomechanical and biometric measurements from one or more of sensors and/or processing units. Microcontroller (18) may receive data from one or more processing units and store the data on non-transitory memory (14). The device may further include a port such as a USB port (24) that may be in electronic communication with microcontroller (18), a battery charging unit (20), and/or non-transitory memory (14). If the device is connected to a central device (such as a personal computer, tablet etc.) via, for example, USB port (24) or via a wireless network protocol, microcontroller (18) may initiate data transfer from the device to the central device (such as a personal computer, tablet etc.), charging of a battery (16) in the device, or a combination of both data transfer and battery charging.

The device may include a communications unit (10) configured to transfer data to and/or from the device. The transfer may occur by a wired communication interface (such as Ethernet, USB), or a wireless communication interface (such as Bluetooth, Wi-Fi, NFC etc.), or a combination of both. A user activity log (64) may be created for each user every time data is recorded and/or when data is transferred to server (52). User activity log (64) may consist of outputs that include summaries of motion history (72), force history (76), temperature history (82), muscle activity history (74), and a synergistic factor analysis (80) that describes how other factors interact with one another.

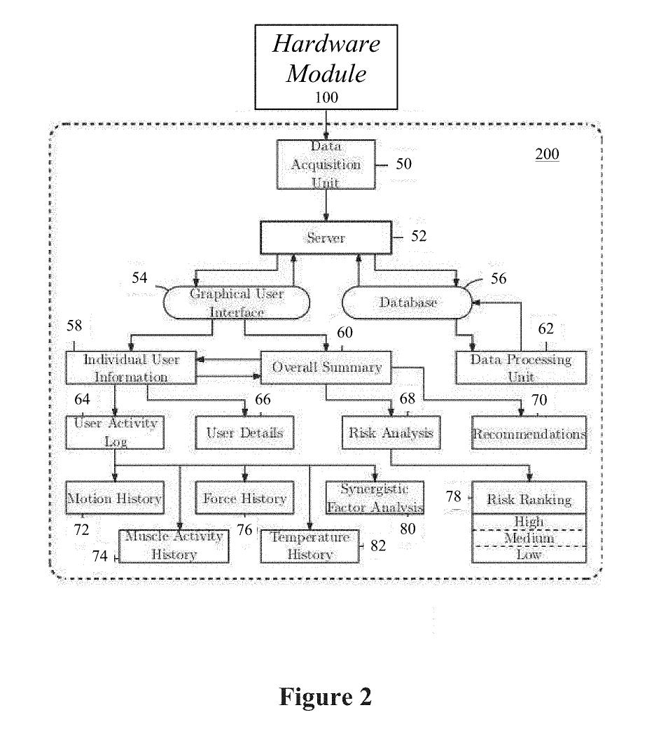

Data generated by hardware module (100) may be processed by hardware module (100), and/or may be transferred to a software module (200). FIG. 2 is a schematic layout of an exemplary software module and exemplary functionality using the data collected from hardware module (100). Communication unit (10) may transfer data to data acquisition unit (50). Data acquisition unit (50) may transfer data to server (52).

Software module (200) may include a database (56) where recorded data may be stored, a data processing unit (62), and a graphical user interface (54) that allows for users to access, analyze and interpret data. Database (56) and a graphical user interface (54) may be accessed on a smart device such as a computer, mobile phone, or tablet. Communications unit (10) may be controlled by microcontroller (18). Microcontroller (18) can send data collected by the processing units (26, 28, 30, 32) via, for example, a USB port (24) that may be connected to the device, wherein data can be transferred by the device to database (56).

Software module (200) may access and process the collected data to provide, for example, metrics relating to user biomechanical behavior and external exposures to the user such as strain, vibration, and repeatedly exceeding limits of motion. Software module (200) may allow for several analytics and visualizations of the collected data that include but are not limited to: total motions performed, isolation of specific habits, exceeding of set limits, total time in a specific posture, force applied to or from a body part, vibration experienced by a body part, temperature of a body part, electric activity in a body part, and other related measures. For example, software module (200) may be used in the work environment to investigate the risk of developing repetitive motion injuries by monitoring risk factors like the number of times a body part is used and the force applied to that body part, as well as the synergy of those risk factors. Software module (200) may highlight areas of potential risk in the collected data for modification to worker environment or behavior.

Server (52) may be, for example, a personal computer, a tablet, or a cloud server. Server (52) may be a web server, and may be in communication with a website and database (56). The data in database (56) may be processed with data processing unit (62) to minimize size and enhance data format for processing and transfer. Server (52) may allow for users to access database (56) through graphical user interface (54).

Graphical user interface (54) may be a website. Graphical user interface (54) may allow for selection of data, and may allow a user to request analyses to be performed on the data. Graphical user interface (54) may allow users to identify the acquired data from hardware module (100) with individual user information (58). Graphical user interface (54) may allow users to view an overall summary (60) of all the collected data in many formats and visuals. The overall summary (60) may allow users to perform a risk analysis (68) for each individual data set that has been recorded.

In one embodiment, a risk analysis (68) may be performed by the device. Risk analysis (68) may be conducted by software stored on non-transitory memory (14). The software may be run by microcontroller (18). In the alternative, risk analysis (68) may be performed by software stored in software module (200). The software may be run by data processing unit (62).

Risk analysis (68) may include a comparison of user data to set standards in the field. For example, in many jobs there are strict guidelines established by the Occupational Safety and Hazards Association (OSHA) that can be input as standards for the risk analysis. If, for example, standards do not exist in the job category of the user, specific values may be input into the risk analysis (68) to set as a standard for comparison.

The device may have an electronic identification scheme to help the software module distinguish one device from another. An RFID unit (12) may be incorporated on the device, and may assign a unique ID (UID) to the device. This UID signal may be transmitted with the biomechanics data. Other options to identify the device include UHF and NFC. The UID based method is especially useful when multiple such devices are working simultaneously. For example, if job-related worker risk assessment is being performed on multiple workers using devices, the RFID unit (12) can be used to distinguish between multiple workers, allowing for simultaneous connection to a software module. In the alternative, if one worker wears multiple devices, the software can use the UID of each device to distinguish between the data transmitted from each device.

The device may include a battery (16) that may power the entire device. The battery may be disposable (such as Nickel oxyhydroxide battery) or rechargeable (such as alkaline, lithium ion), but is not limited to those listed. If the battery is rechargeable, the charging unit (20) may charge the battery (16). Microcontroller (18) monitors and regulates battery (16) usage as well as data transfer via USB port (24).

The device may have one or more user command button(s) to allow the user to initiate and/or control certain functions. Functions of the user command button(s) may include turning on and/or off hardware module (100), communicating with software module (200) through the communications unit (10), initiating data recording from the motion processing unit (28), force processing unit (30), temperature processing unit (32) or muscle activity processing unit (26). For example, pressing a user command button may turn on the device, and pressing the same user command button may initiate recording of data. The user command button(s) may be held for various durations or pressed in different sequences to initiate different functions.

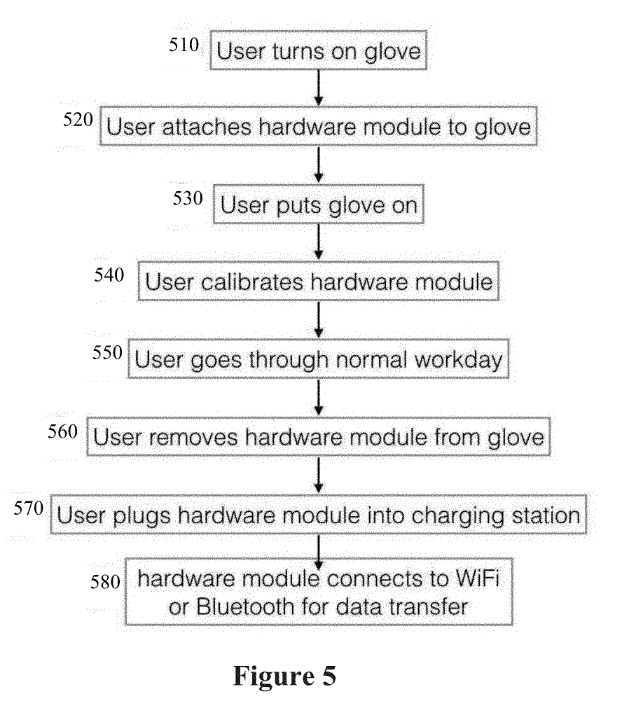

In one embodiment, the device may be used to investigate risk of injury related to a worker's daily behaviors and exposures. FIG. 5 is an example flowchart of how hardware module (100) may be used by an individual to investigate risk of injury relating to their daily behaviors and exposures. At Step (510), a user may turn on the device. At Step (520), the user may attach the device to a hardware module. At Step (530), the user may attach the device to a part of the user's body to be investigated. At Step (540), the device may be calibrated to ensure measurements made by the hardware module are accurate. At Step (550), the user may then proceed with their normal workday or non-workday with the hardware module attached and turned on. At Step (560), when the desired amount of time for monitoring the user has passed, the device may be detached from the hardware module. At Step (570), the hardware module may be charged by plugging into an external power supply. At Step (580), the hardware module may upload data to a database.

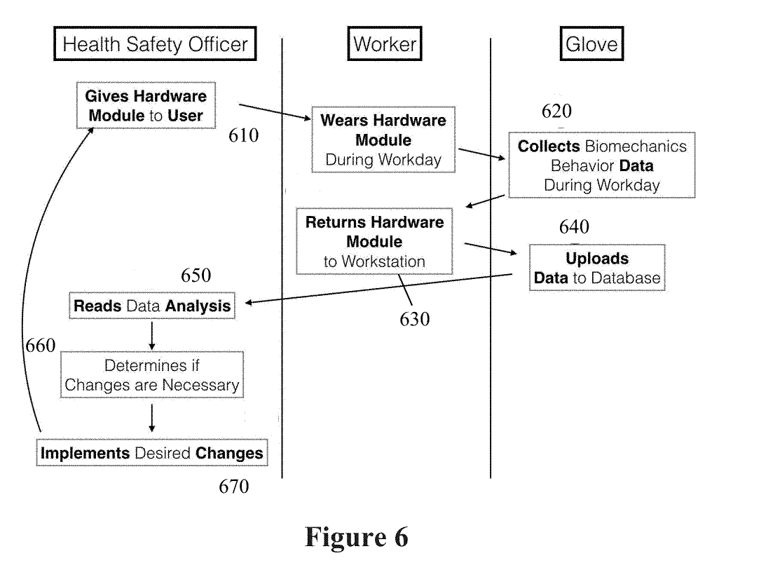

In one embodiment, the device may be used to investigate employee risk of developing a job-related injury. FIG. 6 is an example flowchart showing how a device in accordance with the present invention may be used in a workplace. At Step (610), a health and safety officer may give a worker a device having hardware module (100). At Step (615), the worker may wear the device for a period of time, such as for an entire workday. The hardware module may collect biomechanical behavior and exposure data over the course of the day (620). At the end of the workday, the worker may return the hardware module (630) back to a corresponding workstation. At Step (640), the data may be uploaded from the hardware module to the database. At Step (650), a health and safety officer can then access the data and perform data analysis via a website. At Step (660), the analysis may indicate, for example, whether changes in the workplace should be implemented to reduce worker risk. At Step (670), the health and safety officer may then implement changes to the workplace, such as modification of workplace infrastructure, implementation of new employee breaks or schedules or rotations, elimination of work task, modification of job task, and use of new tool to modify job task.

The device may be mounted to a specific body region of interest on a person for monitoring. The device may attach via one or more methods, depending on the body region, such as by one or more magnets, Velcro, an adhesive, one or more straps, or a sleeve into which the device may slide. Hardware module (100) may be positioned close to the body to optimize measurements made. Hardware module (100) may be adaptable for other users. One hardware module (100) may be suitable for use with any individual. Hardware module (100) may be easily removable after being mounted.

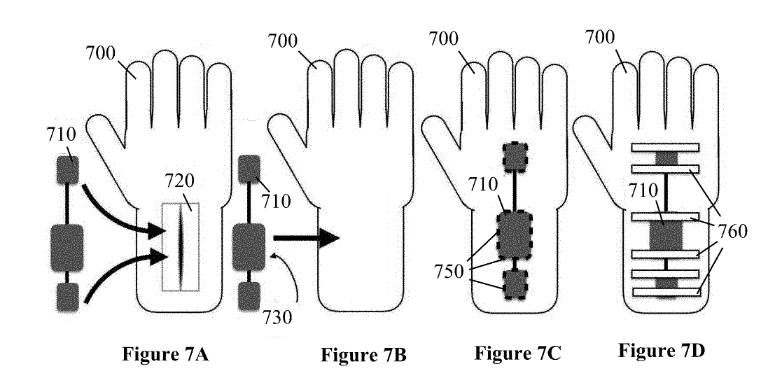

FIGS. 7A-7D depict four mounting methods for the hardware module (710). The device shown in a glove (700), but the device may be any other wearable article. FIG. 7A depicts a pocket mounting method, whereby hardware module (710) may be inserted into pocket (720) on device (700). FIG. 7B depicts a velcro/adhesive mounting method. One side of a velcro (i.e., “hook-and-loop”) connection may be applied to the bottom side (730) of hardware module (710). If a “hook” side is attached to hardware module (710), the “hook” side may connect directly to device (700) if device (700) is made from a material with sufficient “loops.” Otherwise, a corresponding strip of “loops” may be attached to device (700) and the “hook” side may be attached to that strip of “loops.” In the alternative, adhesive may be applied to the bottom side (730) of hardware module (710) and/or to the surface of the device and used to adhere hardware module (710) to device (700). FIG. 7C depicts a stitching mounting method, whereby stitches (750) are used to stitch hardware module (710) to or into the surface of the device. FIG. 7D depicts a strap mounting method, whereby straps (760) are attached to device (700) and are pulled over the hardware module (710) to secure it against the surface of device (700). Hardware module (710) may be attached to device (700) via one or more of the mounting methods depicted in FIGS. 7A-7D.

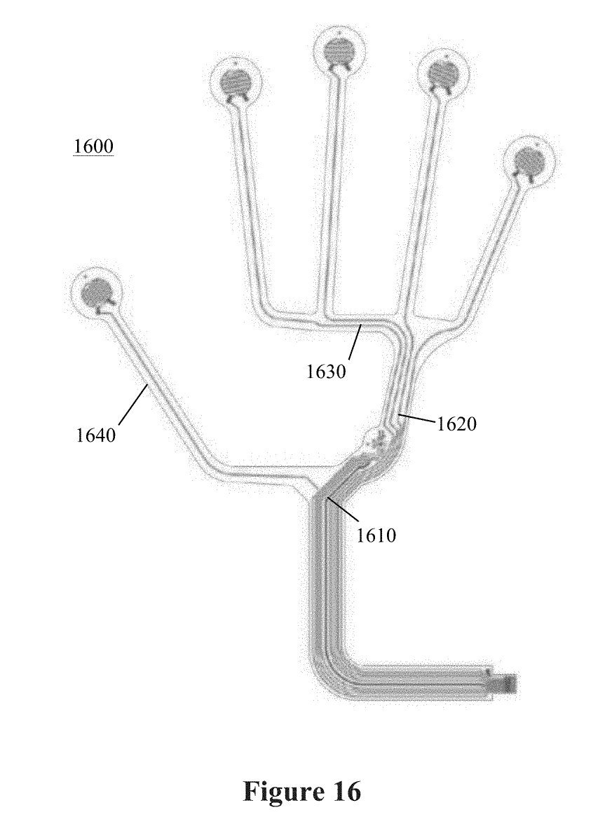

The device may include a flexible printed circuit board (PCB). For example, hardware module (100, 710) may be printed on a flexible PCB. Using a flexible PCB for the hardware module (100) electronics board would minimize restriction of movement, which may be important if hardware module (100) is mounted on a body part that could cause hardware module (100) to bend. FIG. 16 depicts an exemplary PCB (1600) that may be included in a glove in accordance with the present invention. The PCB (1600) may extend from the bottom edge of the palm of the hand (i.e., adjacent to the wrist) (1610), along the side edge of the hand opposite the thumb (1620), and then across the top of the palm (1630) to each of the fingers. The PCB may also extend along the bottom of the palm toward the thumb (1640) and then along the thumb of the glove (1640). The PCB may be attached to the bottom exterior surface of the glove or to the top exterior surface of the glove. To the extent that the PCB is attached to the top exterior surface of the glove, the PCB may wrap around the ends of the glove fingers to attach to sensors at the fingertips of the glove.

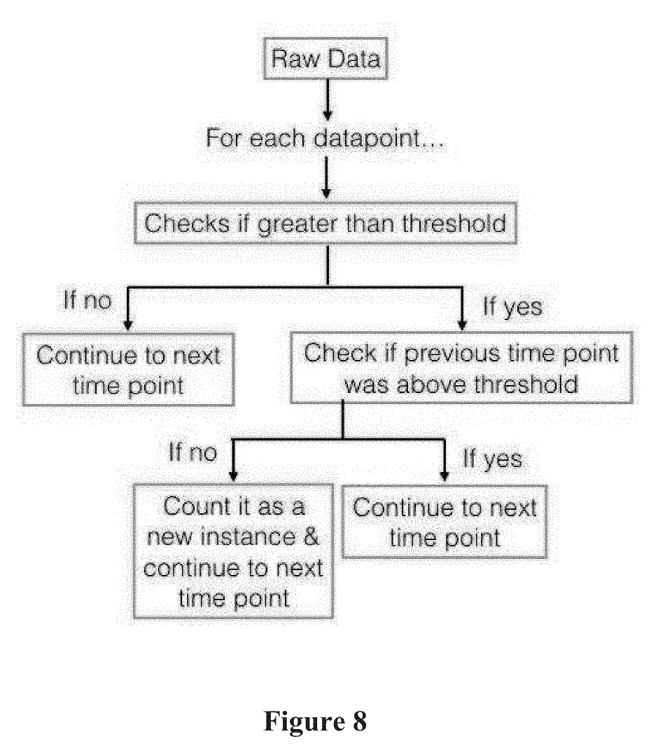

FIG. 8 depicts an example flowchart of a potential risk analysis (68) algorithm. The algorithm may take each sensor data point and compare it to a known or imputed threshold value. If the data point is not above the threshold, the algorithm may continue on to examine the next data point in the user database (56). Whether or not the data point exceeds the threshold set in the algorithm, as well as the magnitude of the motion, is recorded in the database. During data processing, scores may be assigned based on the impact of the risk, with low scores correlating to a small or no additional risk and high scores indicating that the parameter is likely to significantly increase risk factor. To determine the final risk score, these factors may be multiplied together to get a total risk assessment score. The risk analysis (68) may output a final risk ranking (78) for each individual.

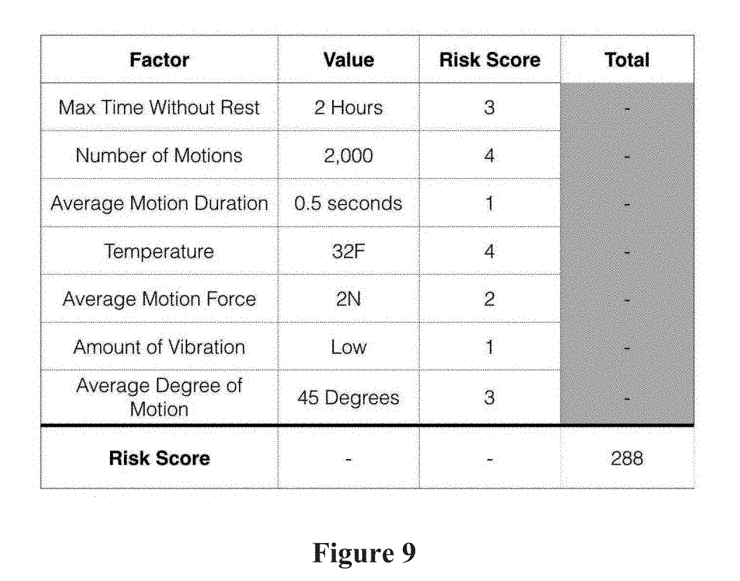

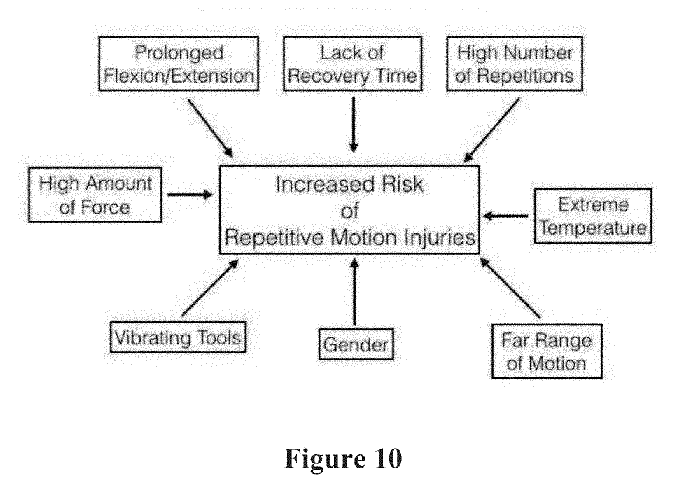

The synergistic factor analysis (80) may allow enhanced understanding of how biomechanical behavior and exposure factors can synergize together to increase worker job-related injury risk. FIG. 9 depicts one example of how synergistic factor analysis (80) may look with several factors being investigated simultaneously, a measured value for each factor being investigated, an assigned risk value for each factor being investigated, and an overall synergistic risk score. For example, when investigating risk of developing repetitive motion injuries it is crucial to investigate the motion itself as well as how much force is being applied in that motion, as both motion and force can synergize to increase the likelihood of an injury developing. FIG. 10 outlines risk factors that can lead to repetitive motion injuries that include but are not limited to prolonged flexion/extension, lack of recovery time, high number of repetitions, extreme temperature, far range of motion, gender, vibrating tools, and high amount of force.

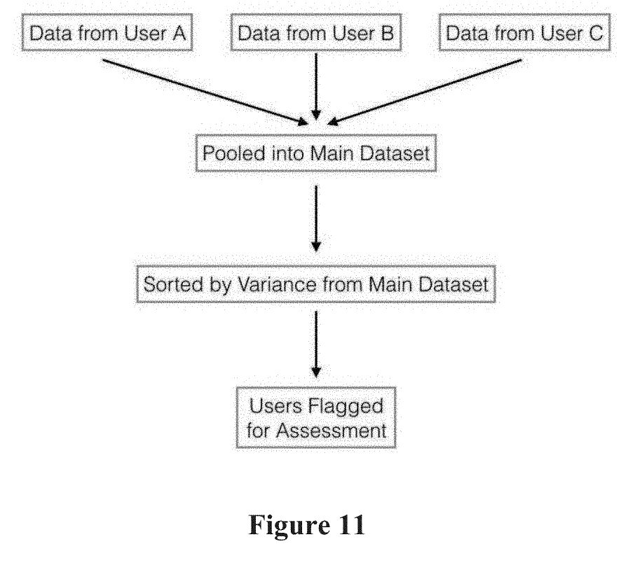

FIG. 11 depicts an exemplary software module receiving data sets from multiple users for comparison simultaneously. Data from each user may be uploaded to the database (56) and may be accessed through the website (54). The data from several users may be pooled into one main data set. Analyses may then be performed on the main data set and each user data set can be compared to the main data set. Each user may then be sorted by the level variance of their activity compared to the main data set. If a user data set is variable from main data set, it may be flagged or highlighted for investigation. For example, an individual investigating worker job-related injury risk may take data collected from employees and pool it together to create a main employee data set.

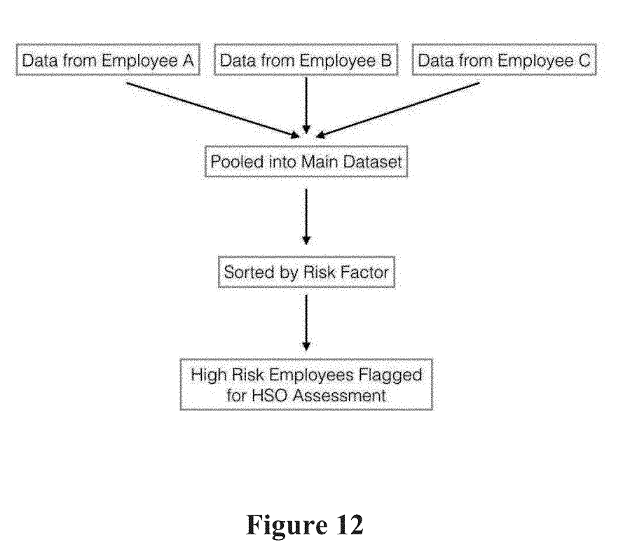

FIG. 12 depicts collected data from several employees being pooled into one main data set. Each employee may be compared to the main data set and/or sorted by their variance from the main data set. A health and safety officer may be notified if an employee's data set varies significantly from the main employee data set.

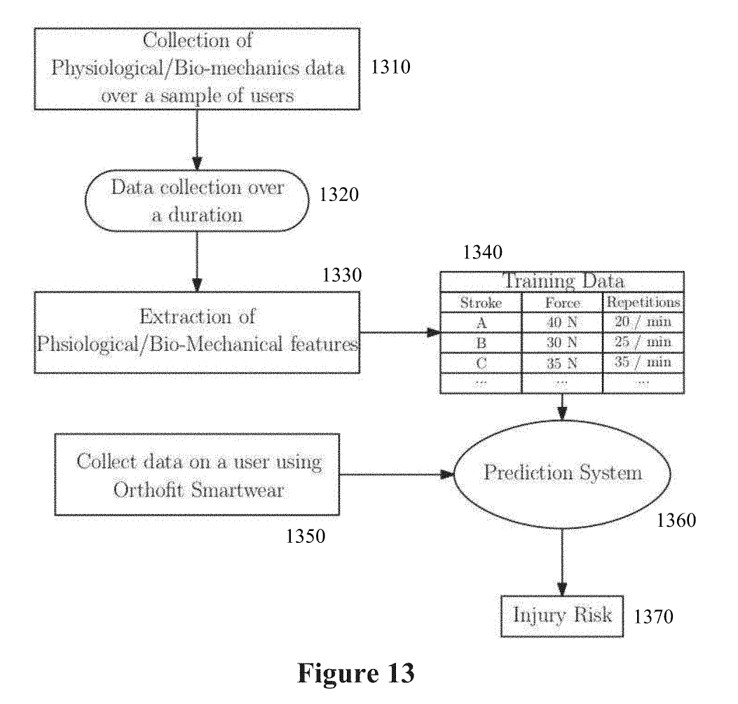

In one embodiment the software module (200) is capable of performing a long-term risk prediction based on user data. FIG. 13 depicts the process of injury prediction based on user data. At Step 1310, a sample of users are provided devices in accordance with the present invention and, at Step 1320, data is collected from those users. The data may include whether, and the extent to which, the users experience injuries. At Step 1330, some or all of the data may be combined to produce a combined data set. At Step 1340, a neural network algorithm and program may generate one or more neural network weights using common machine learning techniques. A neural network function may also be generated using the one or more neural network weights, wherein the neural network function is configured to receive as input data corresponding to sensor data from hardware module (100), and output a probability of whether the an injury is likely to occur. At Step 1350, a new user may be provided a device in accordance with the present invention and sensor data corresponding to the new user is collected from the device. At Step 1360, the sensor data corresponding to the new user may be input to the neural network function. At Step 1370, the neural network may generate a value indicating the extent to which an injury is likely to occur if the new user continues to work in the conditions and in the manner that generated the data in Step 1350.

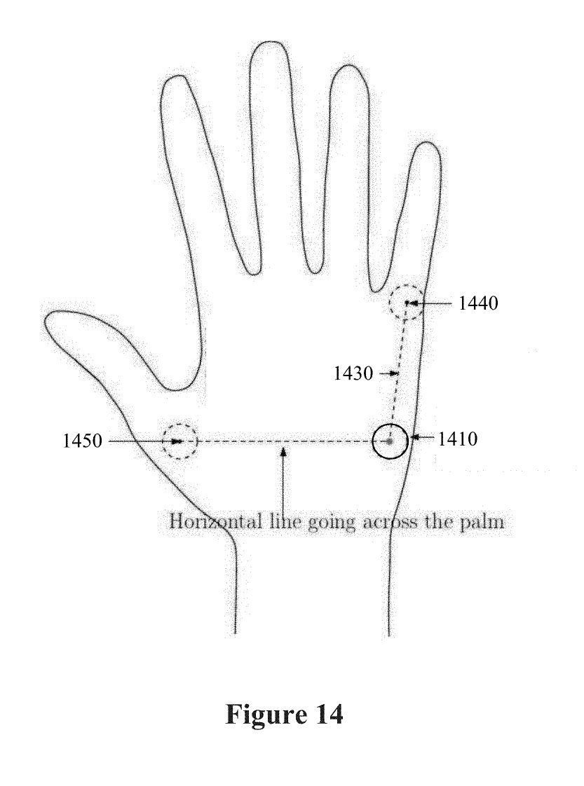

FIG. 14 depicts an exemplary location of an accelerometer (1410) on a device in accordance with the present invention. As shown in FIG. 14, accelerometer (1410) may be located at the intersection of a line drawn horizontal across the bottom edge of the palm, starting from the base of the thumb (1450), and a line (1430) drawn parallel to the edge of the hand opposite the thumb, starting from the base of the pinky finger (1440).

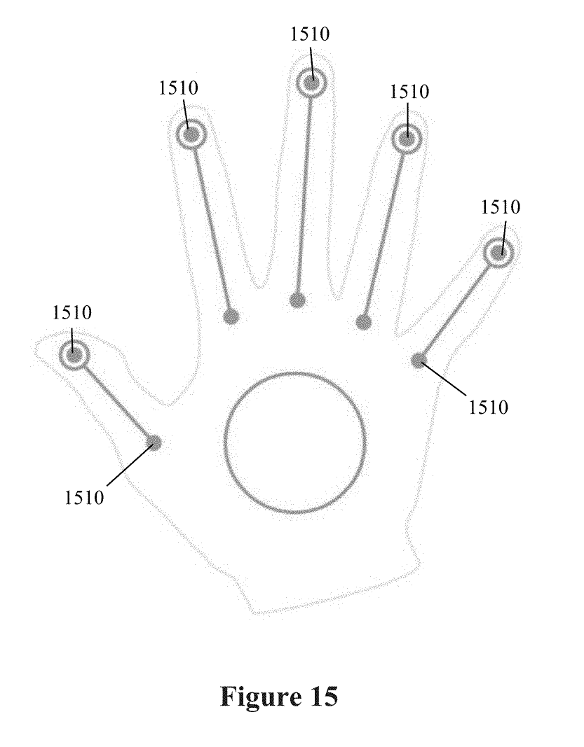

FIG. 15 depicts exemplary locations of pressure sensors (1510) in accordance with the present invention. Pressure sensors may be located at one, some, or all of the fingertips of the device. In addition, or in the alternative, discrete pressure sensors may be located along the center line of one, some, or all of the fingers. For example, a pressure sensor may be located between each knuckle of each finger. In addition, or in the alternative, a pressure sensor may extend continuously along the center line of one, some, or all of the fingers.

While the invention has been described in detail with reference to embodiments for the purposes of making a complete disclosure of the invention, such embodiments are merely exemplary and are not intended to be limiting or represent an exhaustive enumeration of all aspects of the invention. It will be apparent to those of ordinary skill in the art that numerous changes may be made in such details, and the invention is capable of being embodied in other forms, without departing from the spirit, essential characteristics, and principles of the invention. Also, the benefits, advantages, solutions to problems, and any elements that may allow or facilitate any benefit, advantage, or solution are not to be construed as critical, required, or essential to the invention. The scope of the invention is to be limited only by the appended claims.

What is claimed is:

1. A device in the form of a glove having a plurality of fingers for measuring biomechanical factors, comprising:
a printed circuit board;
a first force sensor electronically connected to the printed circuit board;
an electromyogram sensor electronically connected to the printed circuit board;
a temperature sensor electronically connected to the printed circuit board; and
an accelerometer electronically connected to the printed circuit board, wherein the device is in the form of a glove having a plurality of fingers, a thumb, and a palm, and the accelerometer is located at the intersection of a line drawn horizontal across the bottom edge of the palm, and a line drawn parallel to the edge of the glove opposite the thumb, starting from the base of the pinky finger.

2. The device of claim 1, wherein the printed circuit board extends from the bottom edge of the palm of the hand, along the side edge of the hand opposite the thumb, and across the top of the palm to each of the fingers.

3. The device of claim 2, wherein the printed circuit board further extends along the bottom of the palm toward the thumb and along the thumb of the glove.

4. The device of claim 1, wherein the printed circuit board is attached to the bottom exterior surface of the device.

5. The device of claim 1, wherein the printed circuit board is attached to the top exterior surface of the device.

6. The device of claim 5, wherein the printed circuit board wraps around the ends of each finger of the glove.

7. The device of claim 1, wherein the first force sensor is located at the tip of a first finger of the glove.

8. The device of claim 7, further comprising a second force sensor.

9. The device of claim 8, wherein the second force sensor is located at the tip of a second finger of the glove.
